(19) United States
(12) Patent Application Publication
(10) Pub. No.: US 2019/0328322 A1
(43) Pub. Date: Oct. 31, 2019

(54) INFORMATION PROCESSING APPARATUS AND OPERATION METHOD THEREOF

(71) Applicant: FAMILY INADA CO., LTD., Osaka (JP)

(72) Inventor: Nichimu Inada, Osaka (JP)

(21) Appl. No.: 16/233,762

(22) Filed: Dec. 27, 2018

(30) Foreign Application Priority Data
Apr. 27, 2018 (JP) 2018-086148

(51) Int. Cl. A61B 5/00; G16H 20/30; G16H 20/60; A63B 24/00; G09B 19/00

(52) U.S. Cl. CPC A61B 5/4866; G16H 20/30; G16H 20/60; A63B 2220/806; A63B 24/0075; G09B 19/0092; A63B 2225/20; A61B 5/486

(57) ABSTRACT

An information processing apparatus is configured to repeatedly perform, within a predetermined period, a meal managing process of generating meal management data including data indicating an extra calorie within a predetermined period, and an exercise managing process of generating exercise management data including data indicating an amount of deficient exercise within the predetermined period. The extra calorie is calculated based on a total intake calorie of a user for a period from a start point of time of the predetermined period to a point of time when the extra calorie is calculated and total calorie consumption of the user for the period, and the amount of deficient exercise is calculated as an amount of exercise required to consume the extra calorie.

### RELATED APPLICATION

This application claims the benefit of the Convention priority based on Japanese Patent Application No. 2018-086148 filed on Apr. 27, 2018, the contents of which, including the specification, the claims and the drawings, are incorporated herein by reference in their entirety.

### TECHNICAL FIELD

The present disclosure relates to information processing for health care.

### BACKGROUND ART

Japanese Patent Application Publication (kokai) No. 2006-155010 discloses that an intake calorie taken from a meal and calorie consumption consumed by exercise are stored in a database for health care.

### SUMMARY OF THE INVENTION

An information processing apparatus of the present disclosure is configured to repeatedly perform a meal managing process of generating meal management data including data indicating an extra calorie within a predetermined period and an exercise managing process of generating exercise management data including data indicating the amount of deficient exercise within the predetermined period, within the predetermined period. The extra calorie is calculated based on a total intake calorie of a user for a period from a start point of time of the predetermined period to a point of time when the extra calorie is calculated and total calorie consumption of the user for the period, and the amount of deficient exercise is calculated as the amount of exercise required to consume the extra calorie.

An operation method of an information processing apparatus of the present disclosure repeatedly performs a meal managing process of generating meal management data including data indicating an extra calorie within a predetermined period and an exercise managing process of generating exercise management data including data indicating the amount of deficient exercise within the predetermined period, within the predetermined period. The extra calorie is calculated based on a total intake calorie of a user for a period from a start point of time of the predetermined period to a point of time when the extra calorie is calculated and total calorie consumption of the user for the period, and the amount of deficient exercise is calculated as the amount of exercise required to consume the extra calorie.

Further details will be described as embodiments to be described below.

### BRIEF DESCRIPTION OF THE DRAWINGS

FIG. 1 is a diagram showing a network system including a server.

FIG. 2 is a block diagram of a data obtaining unit or circuit.

FIG. 3 is a block diagram of a data generating unit or circuit.

FIG. 4 is a block diagram of a management data generating unit or circuit.

FIG. 5 is a data structure diagram of a management database.

FIG. 6 is a diagram showing the updating of an extra calorie and the amount of deficient exercise.

FIG. 7 is a diagram showing the updating of the extra calorie and the amount of deficient exercise.

### DESCRIPTION OF THE INVENTION

### Outline of Information Processing Apparatus and Operation Method Thereof

An information processing apparatus according to an embodiment is configured to repeatedly perform, within a predetermined period, a meal managing process of generating meal management data including data indicating an extra calorie within the predetermined period, and an exercise managing process of generating exercise management data including data indicating an amount of deficient exercise within the predetermined period. The extra calorie is calculated based on a total intake calorie of a user for a period from a start point of time of the predetermined period to a point of time when the extra calorie is calculated and total calorie consumption of the user for the period. Since the extra calorie is a management index that reflects both an intake calorie taken from a meal and calorie consumption consumed by exercise, it is possible to perform general care with consideration for both the meal and the exercise. The amount of deficient exercise is calculated as an amount of exercise required to consume the extra calorie. Since the amount of deficient exercise is based on the extra calorie, the amount of deficient exercise is also the management index that reflects both the intake calorie taken from a meal and the calorie consumption consumed by the exercise.

It is preferable that the total intake calorie is updated whenever data indicating an intake calorie of the user is obtained within the predetermined period. It is preferable that the total calorie consumption is updated whenever data indicating calorie consumption of the user is obtained within the predetermined period. It is preferable that the extra calorie is calculated whenever at least any one of the total intake calorie and the total calorie consumption is updated.

It is preferable that the extra calorie is calculated based on an appropriate calorie, and the appropriate calorie is an appropriate intake calorie of the user in the point of time when the extra calorie is calculated.

It is preferable that the extra calorie and the amount of deficient exercise are calculated within each of a plurality of predetermined periods, and data indicating the extra calorie and data indicating the amount of deficient exercise in an end point of time of the predetermined period are registered in a management database.

It is preferable that an advice for the user is generated based on at least any one of the meal management data and the exercise management data.

It is preferable that data for an operation of an exercise machine is generated based on at least any one of the meal management data and the exercise management data.

It is preferable that the meal management data includes data indicating a deficient nutrient within the predetermined period, and data for generating a supplement in a supplement generator is generated based on the data indicating the deficient nutrient.

It is preferable that data for an operation of a massage apparatus is generated based on at least any one of the meal management data and the exercise management data.

It is preferable that the meal management data is generated based on meal contents taken by the user, and the meal contents are detected based on a plurality of images obtained by capturing a dining room in different point of times.

It is preferable that a process of detecting a start point of time of meal and a finish point of time of the meal through image processing based on a plurality of images obtained by capturing a dining room is performed, and the images include an image captured in the detected start point of time of the meal and an image captured in the detected finish point of time of the meal.

It is preferable that the meal management data is generated based on meal contents taken by the user, and the meal contents are input from the user with voice.

An operation method of an information processing apparatus according to another embodiment includes repeatedly performing, within a predetermined period, a meal managing process of generating meal management data including data indicating an extra calorie within the predetermined period, and an exercise managing process of generating exercise management data including data indicating an amount of deficient exercise within the predetermined period. The extra calorie is calculated based on a total intake calorie of a user for a period from a start point of time of the predetermined period to a point of time when the extra calorie is calculated and total calorie consumption of the user for the period, and the amount of deficient exercise is calculated as an amount of exercise required to consume the extra calorie. Example of Information Processing Apparatus

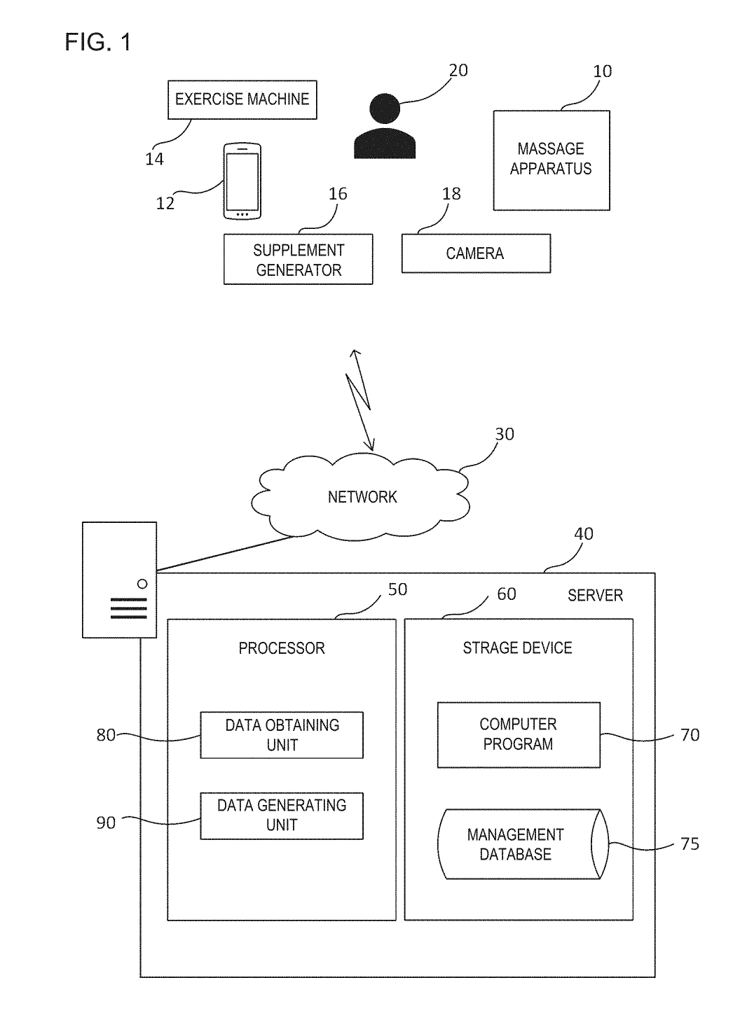

A server (information processing apparatus) 40 shown in FIG. 1 performs information processing for health care of a user 20. The server 40 can communicate with pieces of equipment 10, 12, 14, 16, and 18 used by the user 20 via a network 30. The network 30 is, for example, the Internet and a LAN. The devices used by the user 20 include, for example, a massage apparatus 10, a smartphone 12, an exercise machine 14, a supplement generator 16, and a camera 18.

The server 40 is a computer including a processor 50 and a storage device 60. The processor 50 causes the server 40 to perform information processing for health care by executing a computer program 70 stored in the storage device 60.

The processor 50 functions as a data obtaining unit or circuit 80 by executing the computer program 70. The data obtaining unit 80 obtains data items from the pieces of equipment 10, 12, 14, 16, and 18 of the user.

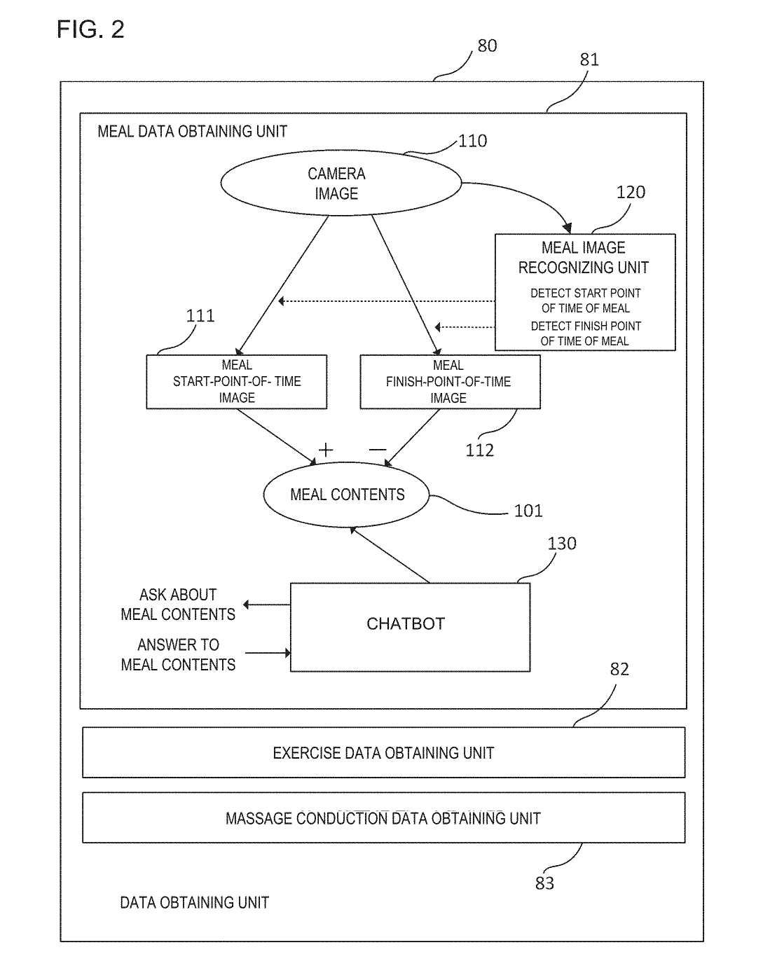

The data items obtained by the data obtaining unit 80 include meal data. As shown in FIG. 2, the data obtaining unit 80 has a function of a meal data obtaining unit or circuit 81. The meal data obtaining unit 81 detects meal contents 101 taken by the user 20, as the meal data.

The meal contents 101 are detected based on an image 110 captured by the camera 18. The camera 18 captures the meal taken by the user 20. The image (moving image) 110 captured by the camera is transmitted to the server 40 from the camera 18. The camera 18 may be attached to the user 20, may be installed on a dining table which is a dining room, or may be installed near the dining table so as to capture the dining table. The camera 18 may be attached to the smartphone 12, or may be attached to glasses of the user 20.

The meal data obtaining unit 81 includes a meal image recognizing unit or circuit 120. The meal image recognizing unit 120 recognizes a foodstuff included in the image 110 through an image recognizing process. The recognizing unit 120 is a recognizer that performs machine learning in order to identify the foodstuff included in the image. The machine learning is, for example, deep learning. The recognizing unit 120 identifies the kind of the foodstuff based on one or a plurality of foodstuff images included in the image 110. The one or plurality of identified foodstuffs is the meal contents. For example, in a case where rice, miso soup, and fried egg are shown in the image 110, the recognizing unit 120 detects that the meal contents include the rice, the miso soup, and the fried egg. In the embodiment, the meal contents 101 are used for calculating an intake calorie and an intake nutrient to be described below.

Although the meal contents may be detected based on one image, the meal contents are also detected based on a plurality of images in the embodiment. For example, in a case where the meal contents are detected based on only a meal start-point-of-time image 111, it is assumed that the user 20 takes all the foodstuffs included in the meal start-point-of-time image 111 as the detected meal contents. Meanwhile, the amount of foodstuffs actually taken by the user 20 can be detected based on the meal start-point-of-time image 111 and a meal finish-point-of-time image 112. That is, a difference in the meal contents detected on both the images 111 and 112 is detected as the amount of meal taken by the user 20. For example, in a case where a bowl of rice is identified on the meal start-point-of-time image 111 and the rice remaining by half in the bowl is identified on the meal finish-point-of-time image 112, the amount of rice remaining by half in the bowl which is a difference in the amount of rice is detected as the amount of rice taken by the user 20.

The recognizing unit 120 has a detection function at a start point of time of the meal and a detection function at a finish point of time of the meal in order to obtain the images 111 and 112. Similarly to the detection of the meal contents, these detection functions are performed through the image recognizing process. The start point of time of the meal is detected as a point of time when a state in which the foodstuffs are not shown on the camera image 110 is changed to a state in which the foodstuffs are shown on the camera image by arranging the foodstuffs on the dining table. The detected camera image 110 at the start point of time of the meal is used as the meal start-point-of-time image 111. For example, the finish point of time of the meal is detected as a point of time immediately before a state in which dishes are shown on the camera image 110 is changed to a state in which the dishes are not shown on the camera by removing the dishes from the dining table. The detected camera image 110 at the finish point of time of the meal is used as the meal finish-point-of-time image 112. The start point of time of the meal and the finish point of time of the meal may be detected based on an operation of the user 20. That is, the user 20 operates the piece of equipment of the user such as the smartphone 12, and thus, the start of the meal or the finish of the meal are notified. Accordingly, the server 40 may detect the start point of time of the meal or the finish point of time of the meal.

The meal contents 101 may be detected at the finish point of time of the meal or may be detected while the user is in the middle of the meal. For example, the meal contents taken in the middle of the meal may be detected from a difference between the meal start-point-of-time image 111 and an in-middle-of meal image. The meal contents taken in the middle of the meal is detected, and thus, it is possible to advise that the user is overeating in the middle of the meal in real time. The camera image 110 at any point of time between the start point of time of the meal and the finish point of time of the meal may be used as the in-middle-of meal image.

In the embodiment, the meal contents may be input from the user 20 with voice. For example, the meal contents with the voice are input by using a chatbot 130. For example, the chatbot 130 asks a question to the user 20 about the meal contents with voice by using the smartphone 12 of the user 20 as a voice input and output device, and receives a voice input as an answer to the meal contents. The question about the meal contents is, for example, “what did you have for breakfast today?”, and the answer to the meal contents is, for example, “rice, miso soup, and fried egg”. The question about the meal contents is transmitted to the smartphone 12 from the meal data obtaining unit 81 of the server 40, and the answer to the meal contents is transmitted to the meal data obtaining unit 81 of the server 40 from the smartphone 12.

The data items obtained by the data obtaining unit 80 include exercise data. As shown in FIG. 2, the data obtaining unit 80 has a function of an exercise data obtaining unit or circuit 82. The exercise data obtaining unit 82 detects the amount of exercise performed by the user 20. For example, the amount of exercise is measured by an activity meter installed on a wearable device of the user 20. The wearable device is, for example, a wearable watch. The amount of exercise may be measured by an application that measures the number of steps of walking or the amount of another exercise, which is installed on the smartphone 12. The measured amount of exercise is transmitted to the exercise data obtaining unit 82 of the server 40 from the smartphone 12. The amount of exercise may be transmitted to the server 40 from the exercise machine 14. The exercise machine 14 is a machine tool with which the user 20 does walking, running, or other exercises, and is, for example, a running machine. The exercise machine 14 has a function of measuring the amount of exercise of the user (a running distance or the number of steps of walking). The amount of exercise measured by the exercise machine 14 is transmitted to the server 40.

The data items obtained by the data obtaining unit 80 includes massage conduction data. As shown in FIG. 2, the data obtaining unit 80 has a function of a massage conduction data obtaining unit or circuit 83. The massage conduction data is data indicating the type and method of the massage conducted for the user by the massage apparatus 20. The massage conduction data is, for example, data indicating a massage course conducted for the user by the massage apparatus 20. The massage conduction data may include data items indicating the type of a massage operation conducted for the user by the massage apparatus 20, a massage treatment part, the strength of the massage, and a massage time. The massage conduction data is transmitted to the massage conduction data obtaining unit 83 of the server 40 from the massage apparatus 10.

As shown in FIG. 1, the processor 50 functions as a data generating unit or circuit 90 by executing the computer program 70. The data generating unit 90 generates various data items.

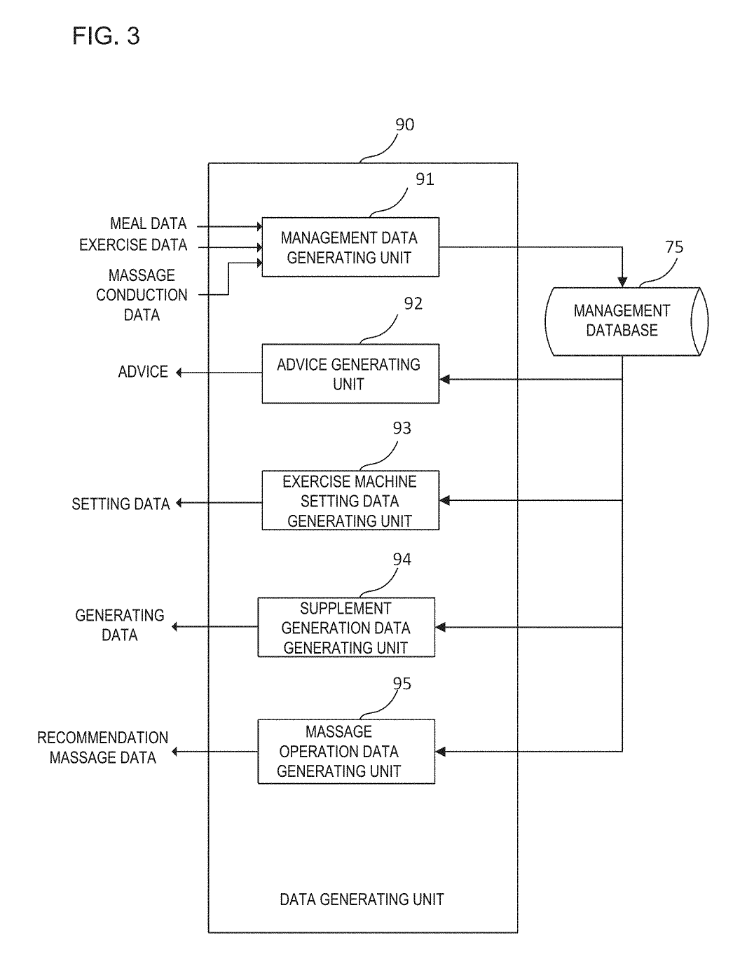

As shown in FIG. 3, the data generating unit 90 has a function of a management data generating unit or circuit 91. The management data generating unit 91 generates management data registered in a management database 75 of the storage device 60. The management data is data for health care of the user 20. The management data is registered in the management database 75 for each user. The management data generating unit 91 generates the management data based on data items (in the embodiment, the meal data, the exercise data, and the massage conduction data) obtained by the data obtaining unit 80. A method of generating the management data will be described below.

The data generating unit 90 has a function of an advice generating unit or circuit 92. The advice generating unit 92 generates an advice on the meal or exercise of the user 20 based on the management data of the user 20 registered in the management database 75. Data indicating the generated advice is transmitted to the smartphone 12 of the user 20 from the server 40.

The data generating unit 90 has a function of an exercise machine setting data generating unit or circuit 93. The exercise machine setting data generating unit 93 generates setting data for user's exercise in the exercise machine 14 to be used by the user 20 based on the management data of the user 20 registered in the management database 75. The generated data is transmitted to the exercise machine 14 from the server 40.

The data generating unit 90 has a function of a supplement generation data generating unit or circuit 94. The supplement generation data generating unit 94 generates data required for preparing a supplement to be generated by the supplement generator 16 based on the management data of the user 20 registered in the management database 75. The generated data is transmitted to the supplement generator 16 from the server 40.

The data generating unit 90 has a function of a massage operation data generating unit or circuit 95. The massage operation data generating unit 95 generates data (recommendation massage data) for the operation of the massage apparatus 10 based on the management data of the user 20 registered in the management database 75. The generated data is transmitted to the massage apparatus 10 from the server 40.

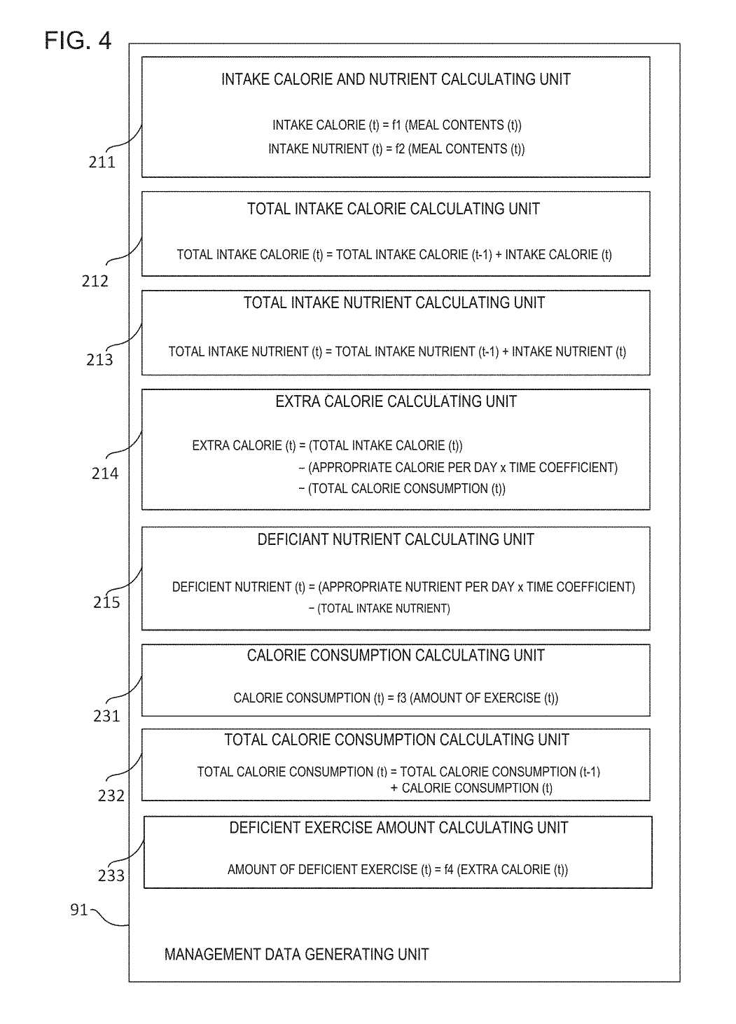

FIG. 4 shows the details of the management data generating unit 91. The management data generating unit 91 includes an intake calorie and nutrient calculating unit or circuit 211. The calculating unit 211 calculates an intake calorie from the meal contents. For example, a table (not shown) in which calories corresponding to the meal contents are registered is registered in the storage device 60, and the intake calorie is calculated by referring to the table. The calculating unit 211 calculates the nutrient taken from the meal contents. For example, a table (not shown) in which nutrients corresponding to the meal contents are registered is registered in the storage device 60, and the intake nutrient is calculated by referring to the table.

In FIG. 4, an intake calorie (t) indicates an intake calorie calculated from meal contents (t) indicated by the meal data obtained by the data obtaining unit 80 of the server 40 in a timing t. The intake nutrient (t) indicates the kind and amount of the intake nutrient calculated from the meal contents (t). The server 40 obtains the meal contents (t) whenever the user 20 takes the meal, the calculating unit 211 calculates the intake calorie (t) and the intake nutrient (t) for the meal whenever the user 20 takes the meal.

The management data generating unit 91 includes a total intake calorie calculating unit or circuit 212. The calculating unit 212 calculates a total intake calorie of the user 20 within a predetermined period. In the embodiment, the predetermined period is a day. The calculating unit 212 repeatedly calculates the total intake calorie whenever the intake calorie (t) is calculated, that is, whenever the user 20 takes the meal during a day. The total intake calorie is a total intake calorie of the user for a period from a start point of time of a day (for example, 12 a.m.) to a point of time t when the total intake calorie is calculated. For example, the total intake calorie (t) in the point of time t is calculated by adding the intake calorie (t) in the point of time t to a total intake calorie (t−1) from the start point of time of a day to a point of time (t−1) earlier than the point of time t. As stated above, the total intake calorie is repeatedly updated for one day which is the predetermined period. The predetermined period is not limited to a day. The predetermined period may be a week, may be ten days, may be a month, or may be any period.

The management data generating unit 91 includes a total intake nutrient calculating unit or circuit 213. The calculating unit 213 calculates a total intake nutrient of the user 20 within a predetermined period (for example, a day). The calculating unit 213 repeatedly calculates the total intake nutrient whenever the intake nutrient (t) is calculated, that is, whenever the user 20 takes the meal for a day which is the predetermined period. The total intake nutrient is a total intake nutrient of the user for a period from a start point of time of a day (for example, 12 a.m.) to a point of time t when the total intake nutrient is calculated. For example, the total intake nutrient (t) in the point of time t is calculated by adding the intake nutrient (t) in the point of time t to a total intake nutrient (t−1) from the start point of time of a day to a point of time (t−1) earlier than the point of time t. As stated above, the total intake nutrient is repeatedly updated for a day which is the predetermined period.

The management data generating unit 91 includes an extra calorie calculating unit or circuit 214. The calculating unit 214 performs a meal managing process of calculating an extra calorie of the user within a predetermined period (for example, a day). The calculating unit 214 repeatedly calculates the extra calorie for a day which is the predetermined period.

The extra calorie indicates a calorie more excessively taken by the user 20 than the calorie consumption of the user 20. Accordingly, the extra calorie may be calculated based on the total intake calorie of the user 20 and a total calorie consumption of the user. The extra calorie is calculated whenever at least any one of the total intake calorie and the total calorie consumption is updated. Accordingly, the user 20 takes the meal or does exercise for a day, and thus, the extra calorie is changed. For example, the user 20 takes the meal, and thus, the extra calorie increases. The user does exercise, and thus, the extra calorie decreases. As a result, an extra calorie in a certain point of time on a certain day indicates an extra intake calorie obtained by adding the intake calorie and the calorie consumption for a period from a start point of time of the certain day to the certain point of time of the certain day. A value of the extra calorie may be a minus value. The minus extra calorie indicates a lack of the calorie.

In the embodiment, the extra calorie is calculated with consideration for an appropriate calorie. That is, in the embodiment, the extra calorie is calculated based on the total intake calorie, the appropriate calorie, and the total calorie consumption, as shown in FIG. 4.

The appropriate calorie is an appropriate intake calorie of the user 20. For example, the appropriate calorie is determined as a calorie corresponding to basal metabolism of the user 20 based on attributes (age, height, weight, and gender) of the user 20. The attributes of the user 20 are specified user information (not shown) registered in the storage device 60.

In the embodiment, a calorie (appropriate calorie per day×time coefficient) obtained by multiplying the appropriate calorie per day by a time coefficient is used as the appropriate calorie to be used in the calculation of the extra calorie. The appropriate calorie per day is an appropriate calorie taken by the user 20 for a day. For example, in the case of a time zone (for example, from 12 a.m. to 10 a.m.) including a point of time of breakfast, 1/3 is used as the time coefficient. In the case of a time zone (for example, from 10 a.m. to 5 p.m.) including a point of time of lunch, 2/3 is used as the time coefficient. In the case of a time zone (for example, from 5 p.m. to 12 a.m.) including a point of time of dinner, 3/3 is used as the time coefficient.

Due to the use of the time coefficient, the appropriate calorie is changed depending on the time zone for a day, and thus, an appropriate value in a point of time when the extra calorie is calculated is obtained. For example, in a case where a point of time when the extra calorie is calculated is within the time zone including the point of time of breakfast, 1/3 of the appropriate calorie per day is an appropriate calorie for calculating the extra calorie. In a case where the point of time when the extra calorie is calculated is within the time zone including the point of time of lunch, 2/3 of the appropriate calorie per day is an appropriate calorie for calculating the extra calorie. In a case where the point of time when the extra calorie is calculated is within the time zone including the point of time of dinner, 3/3 of the appropriate calorie per day is an appropriate calorie for calculating the extra calorie. The time coefficient may be more minutely changed, or may be changed, for example, every hour.

As shown in FIG. 4, in the embodiment, the extra calorie is calculated by subtracting the appropriate calorie in the point of time when the extra calorie is calculated and the total calorie consumption (t) form the total intake calorie (t) in the point of time t when the extra calorie is calculated.

The management data generating unit 91 includes a deficient nutrient calculating unit or circuit 215. The calculating unit 215 calculates the amount of deficient nutrients of the user within a predetermined period (for example, a day). The calculating unit 215 repeatedly calculates deficient nutrients for a day which is the predetermined period.

The deficient nutrient indicates the kind and amount of the nutrient deficient in the meal taken by the user 20. The deficient nutrient is calculated by subtracting the total intake nutrient from an appropriate nutrient. More specifically, as shown in FIG. 4, in the embodiment, the deficient nutrient (t) is calculated by subtracting a total intake nutrient (t) in a point of time t when the deficient nutrient is calculated from the appropriate nutrient in the point of time t when the deficient nutrient is calculated. The appropriate nutrient indicates the kind and amount of the appropriate nutrient to be taken by the user 20. In the embodiment, a nutrient (appropriate nutrient per day×time coefficient) obtained by multiplying the appropriate nutrient per day by a time coefficient is used as the appropriate nutrient to be used in the calculation of the deficient nutrient. The time coefficient may be the same as the time coefficient multiplied by the appropriate calorie per day.

The deficient nutrient is calculated whenever the total intake nutrient is updated. Accordingly, the user takes the meal or skips the meal, and thus, the deficient nutrient is changed. For example, the user takes an appropriate meal, and thus, the deficient nutrient decreases. The user takes a meal with a deficient nutrient or skips the meal, and thus, the deficient nutrient increases. As a result, the deficient nutrient in a certain point of time on a certain day indicates a deficient nutrient for a period from a start point of time of the certain day to the certain point of time on the certain day. A value of the deficient nutrient may be a minus value. The minus deficient nutrient indicates an extra nutrient.

The management data generating unit 91 includes a calorie consumption calculating unit or circuit 231. The calculating unit 231 calculates calorie consumption from the amount of exercise of the user. The calorie consumption is calculated depending on the amount of exercise. In FIG. 4, the amount of exercise (t) indicates the amount of exercise (t) obtained by the data obtaining unit 80 of the server 40 in the timing t. Since the server 40 obtains the amount of exercise (t) whenever the user 20 does exercise, the calculating unit 231 calculates the calorie consumption (t) whenever the user 20 does exercise. Alternatively, since the server 40 obtains the amount of exercise (t) for every exercise unit time (or predetermined time, for example, every hour), the calculating unit 231 calculates the calorie consumption (t) for every exercise unit time. The calorie consumption (t) may be obtained from the exercise machine 14.

The management data generating unit 91 includes a total calorie consumption calculating unit or circuit 232. The calculating unit 232 calculates total calorie consumption of the user 20 within a predetermined period (for example, a day). The calculating unit 232 repeatedly calculates the total calorie consumption whenever the calorie consumption (t) is calculated, that is, whenever the user 20 does exercise or for every exercise unit time for a day which is the predetermined period. The total calorie consumption is total calorie consumption of the user for a period from a start point of time of a day (for example, 12 a.m.) to a point of time t when the total calorie consumption is calculated. For example, the total calorie consumption (t) in the point of time t is calculated by adding the calorie consumption (t) in the point of time t to total calorie consumption (t−1) from the start point of time of a day to a point of time (t−1) earlier than the point of time t. As stated above, the total calorie consumption is repeatedly updated for a day which is the predetermined period.

The total calorie consumption is used in the calculation of the extra calorie in addition to the total intake calorie. The extra calorie is also calculated in a case where the total calorie consumption is updated in addition to a case where the total intake calorie is updated. Accordingly, an extra calorie for a certain point of time for a day may indicate an appropriate value corresponding to the meal for a period from a start point of time of a day to a point of time when the extra calorie is calculated and the content of the exercise.

The management data generating unit 91 includes a deficient exercise amount calculating unit or circuit 233. The calculating unit 233 performs an exercise managing process of calculating the amount of deficient exercise of the user within a predetermined period (for example, a day). The amount of deficient exercise is calculated as the amount of exercise necessary for consuming the extra calorie. The amount of deficient exercise is calculated whenever the extra calorie is calculated. Accordingly, the amount of deficient exercise is calculated whenever at least any one of the total intake calorie and the total calorie consumption is updated. The amount of deficient exercise in a certain point of time for a day may indicate an appropriate value corresponding to the meal for a period from a start point of time of a day to a point of time when the amount of deficient exercise is calculated and the content of the exercise.

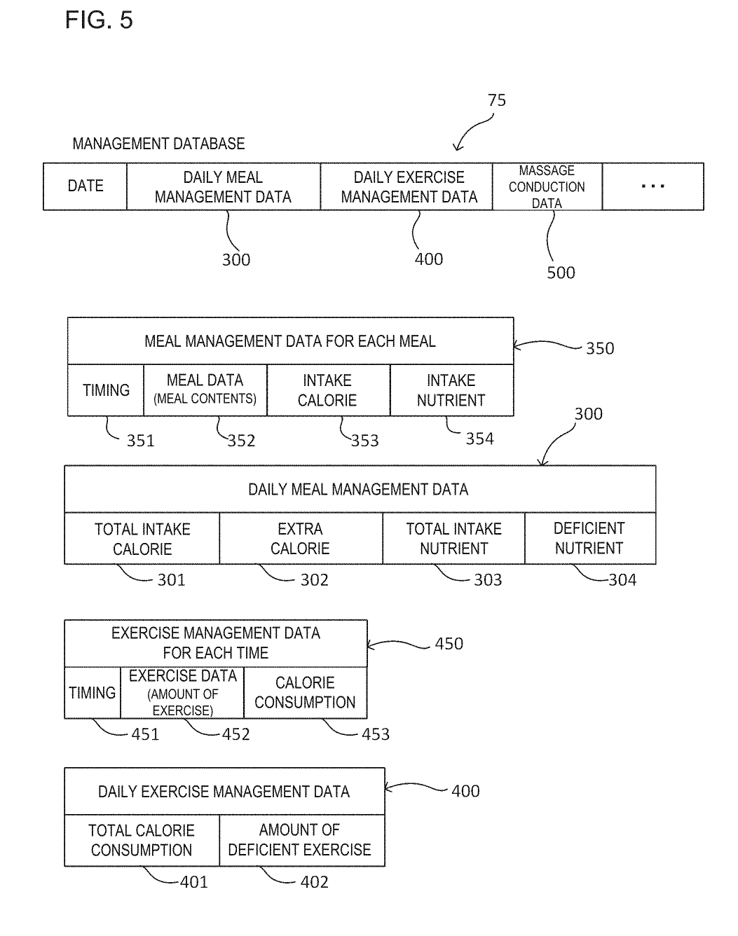

FIG. 5 shows the management database 75 in which various data items (management data items) generated by the management data generating unit 91 of FIG. 4 are registered. The management database 75 manages the management data items of the user for every day (for predetermined period). As shown in FIG. 5, the management data items per day include daily meal management data 300, daily exercise management data 400, and massage conduction data 500 for the day in addition to a region indicating a date.

The daily meal management data 300 has regions of a total intake calorie 301, an extra calorie 302, a total intake nutrient 303, and a deficient nutrient 304. The total intake calorie calculated by the total intake calorie calculating unit 212 is stored in the total intake calorie 301. The extra calorie calculated by the extra calorie calculating unit 214 is stored in the extra calorie 302. The total intake nutrient calculated by the total intake nutrient calculating unit 213 is stored in the total intake nutrient 303. The deficient nutrient calculated by the deficient nutrient calculating unit 215 is stored in the deficient nutrient 304. In a point of time in the middle of the current day, values stored in the regions of the total intake calorie 301, the extra calorie 302, the total intake nutrient 303, and the deficient nutrient 304 are values in the point of time. Values stored in the regions of the total intake calorie 301, the extra calorie 302, the total intake nutrient 303, and the deficient nutrient 304 for the past day are values in an end point of time of every day (for example, 12 a.m.).

The daily exercise management data 400 has regions of total calorie consumption 401 and the amount of deficient exercise 402. The total calorie consumption calculated by the total calorie consumption calculating unit 232 is stored in the total calorie consumption 401. The amount of deficient exercise calculated by the deficient exercise amount calculating unit 233 is stored in the region of the amount of deficient exercise 402. The values stored in the regions of the total calorie consumption 401 and the amount of deficient exercise 402 are values in an end point of time of a day (for example, 12 a.m.).

The massage conduction data related to all massages conducted for the user is stored in massage conduction data 500.

The daily management data may include meal management data 350 for each meal of the day and exercise management data 450 for each hour of a day (or for each exercise) in addition to the data items 300, 400, and 500. The meal management data 350 for each meal includes regions of a time 351 when the user takes the meal, meal data 352 indicating the meal contents, an intake calorie 353 taken from the meal, and an intake nutrient 354 taken from the meal. The exercise management data 450 for each hour (or each exercise) includes regions of a timing 451 when the user does exercise, exercise data 452 indicating the amount of exercise, and calorie consumption 453.

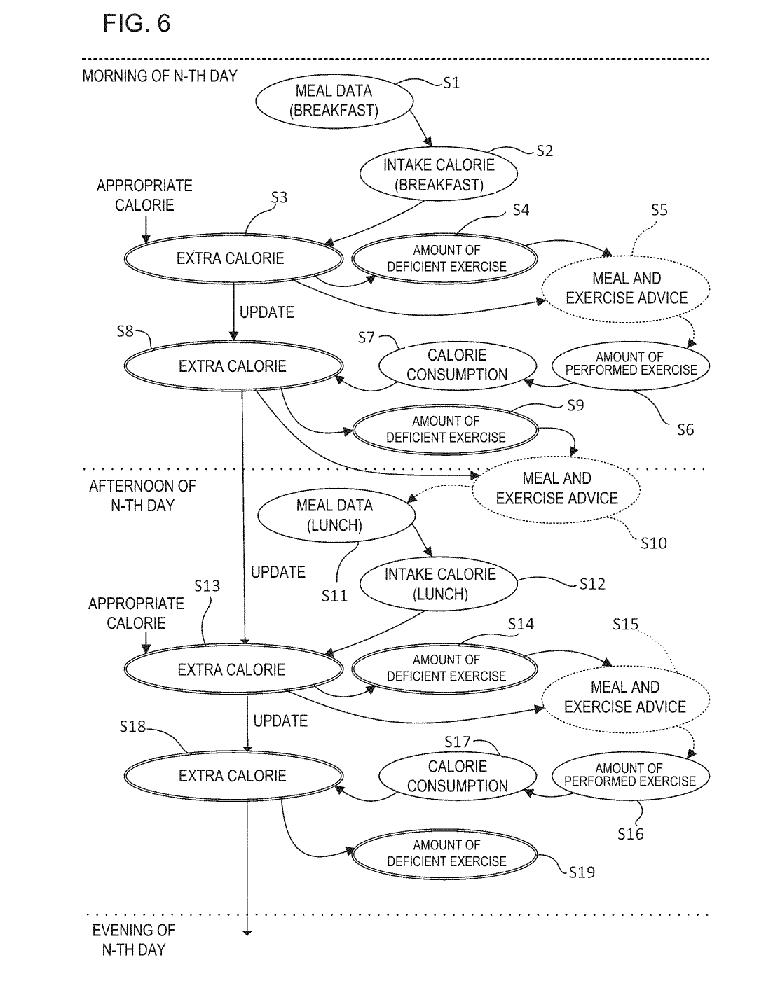

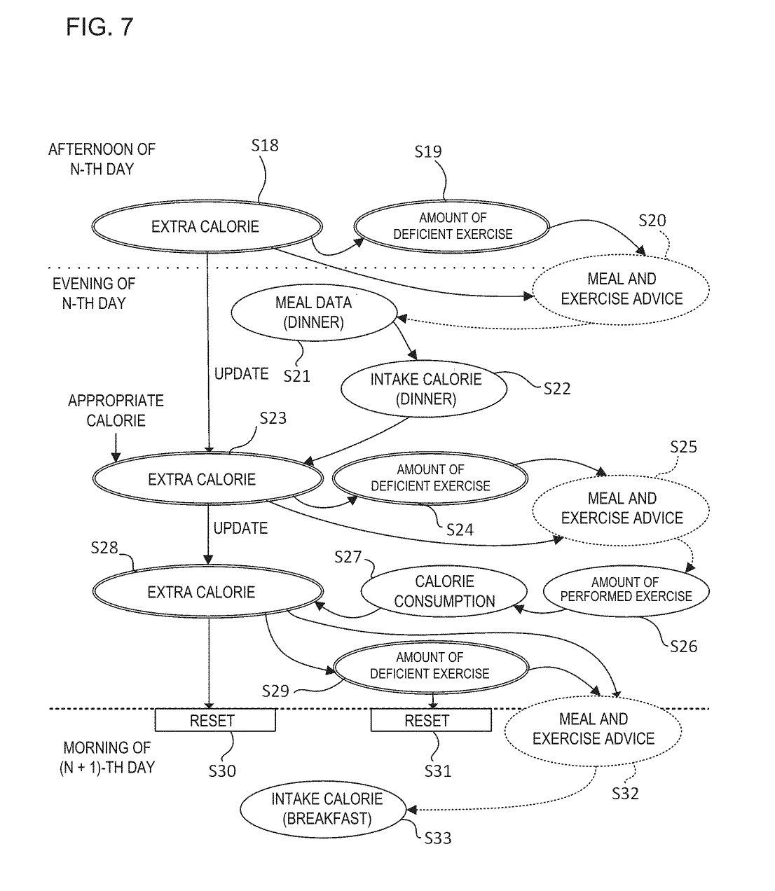

FIGS. 6 and 7 show examples in which the extra calorie and the amount of deficient exercise for a day which is the predetermined period are updated. At the beginning of the day, initial values of the extra calorie, the total intake calorie, the calorie consumption, and the amount of deficient exercise are zero. In a case where the user 20 takes breakfast on the morning of a certain day (N-th day), the meal data obtaining unit 81 obtains the meal data 352 indicating breakfast contents (step S1). Thereafter, the intake calorie and nutrient calculating unit 211 calculates the intake calorie 353 taken from the breakfast from the meal data 352 indicating the breakfast contents (step S2). The intake nutrient 354 taken from the breakfast is also calculated.

The total intake calorie calculating unit 212 uses the intake calorie 353 calculated in step 2 as the total intake calorie 301. The extra calorie calculating unit 214 calculates the extra calorie 302 immediately after the user takes the breakfast from the total intake calorie 301 and the appropriate calorie multiplied by the time coefficient in a point of time when the user takes the breakfast (step S3). The deficient nutrient 304 immediately after the user takes the breakfast is also calculated. The deficient exercise amount calculating unit 233 calculates the amount of deficient exercise 402 immediately after the user takes the breakfast from the extra calorie 302 calculated in step S3 (step S4).

The advice generating unit 92 generates an advice related to the meal or the exercise from the management data including the extra calorie 302 calculated in step S3 and the amount of deficient exercise 402 calculated in step S4 (step S5). For example, the advice is a meal advice indicating that the amount of meal to be taken next by the user needs to be adjusted depending on the value of the extra calorie 302. The advice may be a meal advice indicating that the contents of the meal to be taken next by the user need to be adjusted depending on the value of the deficient nutrient 304. The advice may be an exercise advice indicating the amount of exercise to be recommended to the user depending on the value of the amount of deficient exercise 402. For example, the advice is transmitted to the smartphone 12 of the user 20.

The exercise machine setting data generating unit 93 generates data for setting the amount of exercise with which the user 20 needs to do for the exercise machine 14 from the management data including the amount of deficient exercise 402 calculated in step S4. For example, in a case where the amount of deficient exercise 402 is the amount of exercise corresponding to running 2 km, the exercise machine setting data generating unit 93 generates setting data having the 2-km running as the target amount of exercise, and transmits the generated setting data to the exercise machine 14. Since the target amount of exercise corresponding to the amount of deficient exercise 402 is automatically set for the exercise machine 14, the user 20 can do an appropriate amount of exercise.

The supplement generation data generating unit 94 generates data for generating a supplement to be taken by the user 20 from the management data including the deficient nutrient 304. The data for generating the supplement indicates the kind of the deficient nutrient of the user 20. The data for generating the supplement is transmitted to the supplement generator 16. The supplement generator 16 prepares the supplement for supplementing the deficient nutrient of the user 20 based on the received data, and provides the prepared supplement to the user 20.

The massage operation data generating unit 95 generates data (recommendation massage data) for the operation of the massage apparatus 10 based on the management data of the user. For example, the massage operation data generating unit 95 sets a massage operation to be appropriately performed immediately after the meal in a case where a massage timing is immediately after the meal. For example, the massage operation appropriately performed immediately after the meal is a massage course including an operation for treating meridians that stimulate digestion or an operation for avoiding the treatment of a part that inhibits the digestion. For example, the determination of whether or not the massage timing is immediately after the meal may be performed based on a timing when the user takes the meal included in the management data and a timing when the massage is to be conducted for the user.

The massage operation data generating unit 95 may generate data for the massage operation based on the extra calorie 302 included in the management data. For example, in a case where the extra calorie is a minus value and the calorie is deficient, the massage operation data generating unit may generate data of a massage course including an operation for treating meridians that stimulate appetite.

The massage operation data generating unit 95 may ask the user about their condition by using the chatbot 130 based on the management data of the user 20, and may generate the data for the massage operation. For example, the user is likely to be constipated in the case of exercise deficiency. Thus, in a case where the amount of deficient exercise 402 of the management data is large, the massage operation data generating unit may ask the user about whether or not the user 20 is constipated, and may generate the data of the massage course including an operation for treating meridians suitable for relieving constipation.

The massage operation data generated by the massage operation data generating unit 95 is transmitted to the massage apparatus 10. The massage apparatus 10 may perform the massage operation according to the received data. Accordingly, the user 20 may receive an appropriate massage according to the management data.

The setting data for the exercise machine 14, the data for generating the supplement, and the data for the massage operation may be generated in any timing when the user 20 wants to use these data items (a timing when the user 20 wants to do exercise, a timing when the user 20 wants to generate the supplement, or a timing when the user 20 wants to receive a massage).

In a case where the user 20 does exercise based on the exercise advice (may want to do exercise by using the exercise machine 14) after the breakfast, the amount of exercise of the user is transmitted to the server 40 from the smartphone 12 of the user 20 or the exercise machine 14 (step S6). The calorie consumption calculating unit 231 calculates the calorie consumption 453 from the amount of exercise (step S7).

The total calorie consumption calculating unit 232 uses the calorie consumption 453 calculated in step S7 as the total calorie consumption 401. The extra calorie calculating unit 214 updates the value of the extra calorie 302 from the total calorie consumption 401, the total intake calorie 301 in the current point of time, and the appropriate calorie (step S8). The deficient exercise amount calculating unit 233 updates the amount of deficient exercise 402 based on the extra calorie 302 updated in step S8 (step S9). The extra calorie 302 and the amount of deficient exercise 402 are updated with consideration for the breakfast and the exercise immediately after the breakfast.

The advice generating unit 92 generates the advice related to the meal or the exercise again from the management data including the extra calorie 302 updated in step S8 and the amount of deficient exercise 402 updated in step S9. Since the advice is generated by the updated management data, the appropriate advice is obtained with consideration for the meal and the exercise before a point of time when the advice is generated.

Subsequently, the user 20 may take lunch according to the meal advice. Similarly to the case of the breakfast, in a case where the user takes the lunch, the extra calorie 302 is updated based on the meal data 352 of the lunch (steps S11, S12, and S13), and the amount of deficient exercise 402 is also updated (step S14). The advice related to the meal or the exercise is generated again based on the extra calorie 302 and the amount of deficient exercise 402 after the lunch (step S15). In a case where the user 20 wants to do exercise (may want to do exercise by using the exercise machine 14) based on the exercise advice after the lunch, the extra calorie 302 and the amount of deficient exercise 402 are updated (steps S16, S17, S18, and S19). The advice related to the meal or the exercise is generated again based on the extra calorie 302 and the amount of deficient exercise 402 after the exercise (step S20).

Similarly, the user 20 may take dinner according to the meal advice. In a case where the user takes the dinner, the extra calorie 302 is updated based on the meal data 352 of the dinner (steps S21, S22, and S23), the amount of deficient exercise 402 is also updated (step S24). The advice related to the meal or the exercise is generated again based on the extra calorie 302 and the amount of deficient exercise 402 after the dinner (step S25). In a case where the user 20 wants to do exercise (may want to do exercise by using the exercise machine 14) based on the exercise advice after the dinner, the extra calorie 302 and the amount of deficient exercise 402 are updated (steps S26, S27, S28, and S29).

The extra calorie 302 and the amount of deficient exercise 402 calculated in each point of time for a day are updated in the management database 75, and the values of the extra calorie and the amount of deficient exercise in an end point of time (for example, 12 a.m.) of a day are stored as the extra calorie 302 per day and the amount of deficient exercise 402 per day.

On a (N+1)-th day, the values of the extra calorie, the total intake calorie, the calorie consumption, and the amount of deficient exercise are reset to be zero (steps S30 and S31). Since the extra calorie and the amount of deficient exercise are reset on the morning of the (N+1)-th day, the advice related to the meal or the exercise is generated based on the management data of the previous day (N-th day) or management data items of several days (step S32). The user 20 may take breakfast of the (N+1)-th day according to the meal advice (step S33).

The present invention is not limited to the embodiment, and may be modified in various manners.

What is claimed is:

1. An information processing apparatus configured to repeatedly perform, within a predetermined period, comprising:
a management data generating circuit configured to generate meal management data including data indicating an extra calorie within the predetermined period, the management data generating circuit is further configured to generate exercise management data including data indicating an amount of deficient exercise within the predetermined period, wherein the management generating circuit is configured to repeat to generate the meal management data and the exercise management data within the predetermined period;
the management data generating circuit comprising an extra calorie calculating circuit, a total calorie consumption calculating circuit and a deficient exercise amount calculating circuit,
the extra calorie calculating circuit is configured to calculate the extra calorie based on a total intake calorie of a user for a period from a start point of time of the predetermined period to a point of time when the extra calorie is calculated,
the total calorie consumption calculating circuit is configured to calculate a total calorie consumption of the user for the period, and
the deficient exercise amount calculating circuit is configured to calculate an amount of exercise required to consume the extra calorie.

2. The information processing apparatus according to claim 1,
wherein the extra calorie calculating circuit is configured to update the total intake calorie whenever data indicating an intake calorie of the user is obtained within the predetermined period,
the total calorie consumption calculating circuit is configured to update the total calorie consumption when data indicating calorie consumption of the user is obtained within the predetermined period, and
the extra calorie calculating circuit is configured to calculate the extra calorie when at least one of the total intake calorie and the total calorie consumption is updated.

3. The information processing apparatus according to claim 1,
wherein the extra calorie calculating circuit is configured to calculate the extra calorie based on an appropriate calorie, and the appropriate calorie is an appropriate intake calorie of the user in the point of time when the extra calorie is calculated.

4. The information processing apparatus according to claim 1,
wherein the extra calorie and the amount of deficient exercise are calculated within each of a plurality of predetermined periods, and
data indicating the extra calorie and data indicating the amount of deficient exercise in an end point of time of the predetermined period are registered in a management database.

5. The information processing apparatus according to claim 1,
the apparatus further comprises an advice generating circuit;
wherein the advice generating circuit is configured to generate an advice for the user based on at least one of the meal management data and the exercise management data.

6. The information processing apparatus according to claim 1,
the apparatus further comprises an exercise machine setting data generation circuit;
wherein the exercise machine setting data generating circuit is configured to generate data for an operation of an exercise machine based on at least one of the meal management data and the exercise management data.

7. The information processing apparatus according to claim 1,
wherein the meal management data includes data indicating a deficient nutrient within the predetermined period, and
data for generating a supplement in a supplement generator is generated based on the data indicating the deficient nutrient.

8. The information processing apparatus according to claim 1,
the apparatus further comprises a massage operating data generation circuit;
wherein the massage operating data generating circuit is configured to generate data for an operation of a massage apparatus based on at least any one of the meal management data and the exercise management data.

9. The information processing apparatus according to claim 1,
the apparatus comprises a meal data obtaining circuit that includes a meal image recognizing circuit, the meal image recognizing circuit is configured to capture images of meal contents in different points of times, the meal data obtaining circuit is configured to obtain the meal contents based on the images;
wherein the management data generating circuit is configured to generate the meal management data based on the meal contents.

10. The information processing apparatus according to claim 1,
the apparatus comprises a meal data obtaining circuit that includes a meal image recognizing circuit, the meal image recognizing circuit is configured to capture images of meal contents;
wherein the images include an image captured at a start point of time of the meal and an image captured at a finish point of time of the meal.

11. The information processing apparatus according to claim 1,
the apparatus comprises a meal data obtaining circuit that includes a meal image recognizing circuit, the meal image recognizing circuit is configured to capture images of meal contents, the meal data obtaining circuit is configured to obtain the meal contents based on the images;
wherein the meal management data is generated based on the meal contents, and
the meal contents can be inputted with voice of the user.

12. An operation method of an information processing apparatus, comprising:
repeatedly performing, within a predetermined period,
a meal managing process of generating meal management data including data indicating an extra calorie within the predetermined period, and
an exercise managing process of generating exercise management data including data indicating an amount of deficient exercise within the predetermined period,
wherein the extra calorie is calculated based on a total intake calorie of a user for a period from a start point of time of the predetermined period to a point of time when the extra calorie is calculated and total calorie consumption of the user for the period, and
the amount of deficient exercise is calculated as an amount of exercise required to consume the extra calorie.
